(19) United States
(12) Patent Application Publication
(10) Pub. No.: US 2023/0217953 A1
(43) Pub. Date: Jul. 13, 2023

(54) LOW OSMOLALITY ORAL REHYDRATION SLUSH COMPOSITION

(71) Applicant: ABBOTT LABORATORIES, Abbott Park, IL (US)

(72) Inventors: ELIZABETH MORROW, Columbus, OH (US); MEGAN TERP, Columbus, OH (US); DAVID WOLF, Worthington, OH (US)

(21) Appl. No.: 18/009,580

(22) PCT Filed: Jun. 8, 2021
(86) PCT No.: PCT/US2021/036323

§ 371 Date: Dec. 9, 2022

Related U.S. Application Data
(60) Provisional application No. 63/036,525, filed on Jun. 9, 2020.

(51) Int. Cl. A23G 9/34; A23G 9/04; A23G 9/32; A23L 2/52; A23L 29/25; A23L 29/30; A23L 33/125; A23L 33/16; A61P 3/14; A23L 2/38

(52) U.S. Cl. CPC A23G 9/34; A23G 9/045; A23G 9/325; A23L 2/52; A23L 29/25; A23L 29/30; A23L 33/125; A23L 33/16; A61P 3/14; A23L 2/38

(57) ABSTRACT

A low osmolality oral rehydration slush composition is provided. The oral rehydration slush composition includes: water; a source of carbohydrate; a source of electrolytes; and a source of citrate. The oral rehydration slush composition has an osmolality of 70 mOsm/kg H₂O to 350 mOsm/kg H2O. The oral rehydration slush composition can be used to treat individuals suffering from dehydration.

### CROSS-REFERENCE TO RELATED APPLICATIONS

This application claims priority to and the benefit of U.S. Provisional Patent Application No. 63/036,525, filed on Jun. 9, 2020, the entire content of which is incorporated herein by reference.

### FIELD

The present disclosure relates to oral rehydration compositions. More particularly, the present disclosure relates to a low osmolality oral rehydration slush composition and methods of using the same.

### BACKGROUND

A variety of oral rehydration compositions in a variety of product forms, such as liquids, powders, and tablets, are currently available to consumers. Oral rehydration compositions assist in rehydrating an individual after there has been a substantial loss of fluid, resulting in dehydration. The dehydration may be caused by strenuous exercise, physical exertion, or sports activities, or may be caused by fever, diarrhea, vomiting, or combinations thereof.

A product form popular with children and adults alike is the frozen slush beverage, commonly referred to as a slushy, slushie, or slushee. However, typical frozen slush beverage products are not suitable for oral rehydration therapy due to the high osmolality of such frozen slush beverage products. Accordingly, there remains a need for a frozen slush beverage product that can be used to rehydrate individuals suffering from dehydration or otherwise require hydration.

### SUMMARY

Disclosed herein is an oral rehydration slush composition that can be used to rehydrate individuals suffering from dehydration. The oral rehydration slush composition has a low osmolality which renders the slush composition efficacious for oral rehydration therapy.

In accordance with the present disclosure, an oral rehydration slush composition is provided. The oral rehydration slush composition includes: water; a source of carbohydrate; a source of electrolytes; and a source of citrate. The oral rehydration slush composition has an osmolality of 70 mOsm/kg H₂O to 350 mOsm/kg H₂O.

In accordance with the present disclosure, an oral rehydration slush composition is provided. The oral rehydration slush composition includes: water; dextrose; 15 mEq/L to 120 mEq/L of sodium; 2 mEq/L to 60 mEq/L of potassium; 25 mEq/L to 110 mEq/L of chloride; and 5 mmol/L to 40 mmol/L of citrate. The oral rehydration slush composition has an osmolality of 70 mOsm/kg H₂O to 350 mOsm/kg H₂O.

### BRIEF DESCRIPTION OF THE DRAWING

FIG. 1 is a graph of D90 particle size data of slush compositions during the freezing process in accordance with the Example of the present disclosure.

### DETAILED DESCRIPTION

Disclosed herein is an oral rehydration slush composition. While the present disclosure describes certain embodiments of the oral rehydration slush composition in detail, the present disclosure is to be considered exemplary and is not intended to be limited to the disclosed embodiments. Also, certain elements or features of embodiments disclosed herein are not limited to a particular embodiment, but instead apply to all embodiments of the present disclosure.

The terminology as set forth herein is for description of the embodiments only and should not be construed as limiting the disclosure as a whole. All references to singular characteristics or limitations of the present disclosure shall include the corresponding plural characteristic or limitation, and vice versa, unless otherwise specified or clearly implied to the contrary by the context in which the reference is made. Unless otherwise specified, “a,” “an,” “the,” and “at least one” are used interchangeably. Furthermore, as used in the description and the appended claims, the singular forms “a,” “an,” and “the” are inclusive of their plural forms, unless the context clearly indicates otherwise.

To the extent that the term “includes” or “including” is used in the description or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. Furthermore, when the phrase “one or more of A and B” is employed it is intended to mean “only A, only B, or both A and B.” Similarly, when the phrases “at least one of A, B, and C” or “at least one of A, B, C, and combinations thereof” are employed, they are intended to mean “only A, only B, only C, or any combination of A, B, and C” (e.g., A and B; B and C; A and C; A, B, and C).

The oral rehydration slush composition of the present disclosure can comprise, consist of, or consist essentially of the essential elements of the disclosure as described herein, as well as any additional or optional element or feature described herein or which is otherwise useful in slush type beverages and/or oral rehydration therapy applications.

The oral rehydration slush composition of the present disclosure may be substantially free of any optional or selected ingredient or feature described herein, provided that the remaining composition still contains all of the required ingredients or features as described herein. In this context, and unless otherwise specified, the term “substantially free” means that less than a functional amount of the optional or selected ingredient is present, which is typically less than 0.1% by weight, and also including zero percent by weight of such optional or selected ingredient.

All percentages, parts, and ratios as used herein are by weight of the total composition unless otherwise specified. All such weights as they pertain to listed ingredients are based on the active level and, therefore, do not include solvents, by-products, or other components that may be included in commercially available materials, unless otherwise specified.

All ranges and parameters, including but not limited to percentages, parts, and ratios, disclosed herein are understood to encompass any and all sub-ranges assumed and subsumed therein, and every number between the endpoints. For example, a stated range of “1 to 10” should be considered to include any and all sub-ranges beginning with a minimum value of 1 or more and ending with a maximum value of 10 or less (e.g., 1 to 6.1, or 2.3 to 9.4), and to each integer (1, 2, 3, 4, 5, 6, 7, 8, 9, and 10) contained within the range.

Any combination of method or process steps as used herein may be performed in any order, unless otherwise specified or clearly implied to the contrary by the context in which the referenced combination is made.

The term “about” as used herein means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by 10%.

An equivalent (Eq) is the amount of a substance that can react with one mole of a counter-ion carrying a unit charge (+1 or −1), such as hydrogen (H+) or hydroxide (OH−). In other words, the “equivalent” part of this term, then, means equivalent in terms of chemical valence, not in terms of sheer mass. A milliequivalent (mEq) is one thousandth of an equivalent (Eq). Milliequivalents may be converted to milligrams by multiplying mEq by the atomic weight of the mineral and then dividing that number by the valence of the mineral.

The terms “slush” and “slush composition” are used interchangeably herein and refer to an aqueous beverage containing ice crystals and water soluble ingredients. It should be understood that the slush composition may be provided in powder form, which powder is intended to be added to and mixed with water and then frozen to provide a ready-to-eat slush composition.

Conventional slush compositions are typically formulated with a significant amount (i.e., more than 10% by weight) of additives, which lowers the freezing point of the slush composition and provides the slush composition with a soft, partially unfrozen texture with small, discrete, orally-manipulatable ice crystals. Typical additives used in conventional slush compositions include sugar, high fructose corn syrup, glycerol, monopropylene glycol, colorants, flavorants, and various combinations of the foregoing. However, the significant amount of additives used to achieve the desired slush texture and consistency in conventional slush compositions results in a high product osmolality (e.g., 700 mOsm/kg H₂O to 1,500 mOsm/kg H₂O).

Osmolality is a major factor in the efficacy of oral rehydration therapy, with an osmolality of less than 300 mOsm/kg H₂O considered as ideal. Thus, the high product osmolality associated with conventional slush compositions renders them unsuitable for use in oral rehydration therapy. Moreover, formulating a slush composition with the desired slush texture and consistency while also achieving a low osmolality (e.g., less than 350 mOsm/kg H₂O, preferably less than 300 mOsm/kg H₂O) such that the slush composition is suitable for oral rehydration therapy has heretofore proven elusive.

It has now been discovered that a slush composition suitable for oral rehydration therapy (referred to herein as an “oral rehydration slush composition”) while also having the desired slush texture and consistency of a conventional slush composition can be formulated without using a high sugar content or organic compounds used as freezing point depression agents that are classified as alcohols such as glycerol or monopropylene glycol. In particular, it was discovered that the use of a gum system can achieve an oral rehydration slush composition having a slush texture and consistency similar to that of a conventional slush composition.

In one aspect of the present disclosure, an oral rehydration slush composition is provided. The oral rehydration slush composition comprises: water; a source of carbohydrate; a source of electrolytes; and a source of citrate. In addition, the oral rehydration slush composition of the present disclosure has an osmolality of 70 mOsm/kg H₂O to 350 mOsm/kg H₂O. Because of the low osmolality, the oral rehydration slush composition of the present disclosure can be used to treat individuals suffering from dehydration or otherwise in need of hydration.

The oral rehydration slush composition of the present disclosure includes water. The water serves as a solvent for dissolving other components of the oral rehydration slush composition. In addition, the water crystallizes under freezing conditions (e.g., temperatures of less than or equal to 0° C.) to form ice crystals. Furthermore, the water functions to rehydrate individuals who consume the oral rehydration slush composition. In embodiments, the oral rehydration slush composition comprises from 90 wt % to 98 wt % water. In embodiments, the oral rehydration slush composition comprises from 94 wt % to 97.5 wt % water. In embodiments, the oral rehydration slush composition comprises from 94.5 wt % to 97 wt % water. In embodiments, the oral rehydration slush composition comprises from 95 wt % to 96.5 wt % water.

Alternatively, the oral rehydration slush composition of the present disclosure may be characterized based on its solids content. The term “total solids content” as used herein, unless otherwise specified, refers to the weight percentage of all components other than water (including ice), regardless of whether the other components are in fact solids or liquid, based on the total weight of the oral rehydration slush composition. In embodiments, the oral rehydration slush composition has a total solids content of 2 wt % to 10 wt %. In embodiments, the oral rehydration slush composition has a total solids content of 2.5 wt % to 6 wt %. In embodiments, the oral rehydration slush composition has a total solids content of 3 wt % to 5.5 wt %. In embodiments, the oral rehydration slush composition has a total solids content of 3.5 wt % to 5 wt %.

The oral rehydration slush composition of the present disclosure includes a source of carbohydrate. Typically, an oral rehydration composition includes a source of carbohydrate, most often a simple sugar such as dextrose, to improve absorption of electrolytes present in the oral rehydration composition. The source of carbohydrate present in the oral rehydration slush composition of the present disclosure may be a simple carbohydrate and/or a complex carbohydrate, including monosaccharides, disaccharides, oligosaccharides, and polysaccharides.

In embodiments, the source of carbohydrate present in the oral rehydration slush composition comprises dextrose, fructooligosaccharides, human milk oligosaccharides (e.g., 6′-sialyllactose, 3′-sialyllactose, 2′-fucosyllactose, 3-fucosyllactose, lacto-N-tetraose, lacto-N-neotetraose, lacto-N-fucopentaose), dextrose polymers, corn syrup, high fructose corn syrup, sucrose, maltodextrin, isomaltulose, digestion-resistant maltodextrin, lactose, maltose, amylose, glycogen, galactose, allose, altrose, mannose, gulose, idose, talose, ribose, arabinose, lyxose, ribose, xylose, erythrose, threose, and combinations thereof. In embodiments, the source of carbohydrate present in the oral rehydration slush composition comprises dextrose. In embodiments, the source of carbohydrate present in the oral rehydration slush composition consists of dextrose.

In embodiments, a source of carbohydrate is present in the oral rehydration slush composition in an amount of 0.5 wt % to 5 wt %, based on the total weight of the oral rehydration slush composition. In embodiments, a source of carbohydrate is present in the oral rehydration slush composition in an amount of 1 wt % to 4 wt %, based on the total weight of the oral rehydration slush composition. In embodiments, a source of carbohydrate is present in the oral rehydration slush composition in an amount of 1.5 wt % to 3.75 wt %, based on the total weight of the oral rehydration slush composition. In embodiments, a source of carbohydrate is present in the oral rehydration slush composition in an amount of 2 wt % to 3.75 wt %, based on the total weight of the oral rehydration slush composition. In embodiments, a source of carbohydrate is present in the oral rehydration slush composition in an amount of 2.5 wt % to 3.5 wt %, based on the total weight of the oral rehydration slush composition. In embodiments, a source of carbohydrate is present in the oral rehydration slush composition in an amount of 2.75 wt % to 3.25 wt %, based on the total weight of the oral rehydration slush composition.

In embodiments, the oral rehydration slush composition comprises dextrose in an amount of 0.5 wt % to 5 wt %, based on the total weight of the oral rehydration slush composition. In embodiments, the oral rehydration slush composition comprises dextrose in an amount of 0.75 wt % to 4 wt %, based on the total weight of the oral rehydration slush composition. In embodiments, the oral rehydration slush composition comprises dextrose in an amount of 1 wt % to 3.75 wt %, based on the total weight of the oral rehydration slush composition. In embodiments, the oral rehydration slush composition comprises dextrose in an amount of 1.5 wt % to 3.5 wt %, based on the total weight of the oral rehydration slush composition. In embodiments, the oral rehydration slush composition comprises dextrose in an amount of 2 wt % to 3.25 wt %, based on the total weight of the oral rehydration slush composition.

The oral rehydration slush composition of the present disclosure includes a source of electrolytes. As one of skill in the art knows, electrolytes are substances that ionize or dissociate to produce an electrically conductive solution when dissolved in a polar solvent, such as water. Electrolytes are important from a physiological standpoint, as they regulate nerve and muscle function, hydrate the body, balance blood acidity and pressure, and help rebuild damaged tissue. The primary electrolytes found in the human body include sodium, potassium, chloride, bicarbonate, calcium, magnesium, and phosphate. For purposes of the present disclosure, the term “electrolytes” refers to ions of sodium, potassium, chloride, magnesium, calcium, zinc, and so forth.

In embodiments, the source of electrolytes in the oral rehydration slush composition of the present disclosure comprises one or more of a source of sodium, a source of potassium, and a source of chloride. In embodiments, the source of electrolytes in the oral rehydration slush composition of the present disclosure comprises a source of sodium, a source of potassium, and a source of chloride.

In embodiments, the source of sodium present in the oral rehydration slush composition provides sodium ions. Exemplary sources of sodium suitable for use in the oral rehydration slush composition of the present disclosure include, but are not limited to, sodium chloride, sodium citrate, sodium phosphate, sodium carbonate, sodium bicarbonate, sodium hydroxide, and combinations thereof. In embodiments, the source of sodium comprises a sodium salt, including one or more of those listed above. In embodiments, the source of sodium present in the oral rehydration slush composition of the present disclosure comprises sodium chloride and sodium citrate.

The quantity of sodium present in the oral rehydration slush composition may vary widely. In general, the quantity of sodium ions present in the oral rehydration slush composition is not less than 15 mEq/L. In embodiments, the oral rehydration slush composition of the present disclosure comprises a source of sodium in an amount sufficient to provide 15 mEq to 120 mEq of sodium per liter of the oral rehydration slush composition. In embodiments, the oral rehydration slush composition of the present disclosure comprises a source of sodium in an amount sufficient to provide 20 mEq to 100 mEq of sodium per liter, including from 25 mEq to 80 mEq of sodium per liter, from 30 mEq to 70 mEq of sodium per liter, from 35 mEq to 60 mEq of sodium per liter, and also including from 40 mEq to 50 mEq of sodium per liter of the oral rehydration slush composition.

In embodiments, the source of potassium present in the oral rehydration slush composition provides potassium ions. Exemplary sources of potassium suitable for use in the oral rehydration slush composition of the present disclosure include, but are not limited to, potassium citrate, potassium chloride, potassium phosphate, potassium carbonate, potassium bicarbonate, potassium hydroxide, and combinations thereof. In embodiments, the source of potassium comprises a potassium salt, including one or more of those listed above. In embodiments, the source of potassium present in the oral rehydration slush composition of the present disclosure comprises potassium citrate.

The quantity of potassium present in the oral rehydration slush composition may vary widely. In embodiments, the oral rehydration slush composition of the present disclosure comprises a source of potassium in an amount sufficient to provide 2 mEq to 60 mEq of potassium per liter of the oral rehydration slush composition. In embodiments, the oral rehydration slush composition of the present disclosure comprises a source of potassium in an amount sufficient to provide 5 mEq to 50 mEq of potassium per liter, including from 10 mEq to 40 mEq of potassium per liter, from 15 mEq to 30 mEq of potassium per liter, and also including from 15 mEq to 25 mEq of potassium per liter of the oral rehydration slush composition.

In embodiments, the source of chloride present in the oral rehydration slush composition provides chloride ions. Exemplary sources of chloride suitable for use in the oral rehydration slush composition of the present disclosure include, but are not limited to, sodium chloride, potassium chloride, calcium chloride, magnesium chloride, and combinations thereof. In embodiments, the source of chloride comprises a chloride salt, including one or more of those listed above. In embodiments, the source of chloride present in the oral rehydration slush composition of the present disclosure comprises sodium chloride.

The quantity of chloride present in the oral rehydration slush composition may vary widely. In embodiments, the oral rehydration slush composition of the present disclosure comprises a source of chloride in an amount sufficient to provide 25 mEq to 110 mEq of chloride per liter of the oral rehydration slush composition. In embodiments, the oral rehydration slush composition of the present disclosure comprises a source of chloride in an amount sufficient to provide 25 mEq to 90 mEq of chloride per liter, including from 25 mEq to 75 mEq of chloride per liter, from 25 mEq to 50 mEq of chloride per liter, and also including from 30 mEq to 40 mEq of chloride per liter of the oral rehydration slush composition.

The oral rehydration slush composition of the present disclosure also includes a source of citrate. When the oral rehydration slush composition is consumed, the source of citrate functions as a base to help replace diarrheal losses. Citrate is metabolized to an equivalent amount of bicarbonate, the base in the blood that helps maintain acid-base balance. Exemplary sources of citrate suitable for use in the oral rehydration slush composition of the present disclosure include, but are not limited to, citric acid, potassium citrate, and sodium citrate. In embodiments, the source of citrate present in the oral rehydration slush composition comprises citric acid, potassium citrate, and sodium citrate.

The quantity of citrate present in the oral rehydration slush composition may vary widely. In embodiments, the oral rehydration slush composition of the present disclosure comprises a source of citrate in an amount sufficient to provide 5 mmol to 40 mmol of citrate per liter of the oral rehydration slush composition. In embodiments, the oral rehydration slush composition of the present disclosure comprises a source of citrate in an amount sufficient to provide 10 mmol to 40 mmol of citrate per liter, including from 20 mmol to 40 mmol of citrate per liter, and also including from 30 mmol to 40 mmol of citrate per liter of the oral rehydration slush composition.

It should be understood that certain ingredients of the oral rehydration slush composition of the present disclosure may satisfy more than one category of ingredients present in the oral rehydration slush composition. As one example, sodium citrate is a source of electrolytes (i.e., sodium) and is also a source of citrate. As another example, sodium chloride is source of sodium and is also a source of chloride.

The oral rehydration slush composition of the present disclosure has an osmolality of 70 mOsm/kg H₂O to 350 mOsm/kg H₂O. The osmolality of the oral rehydration slush composition may be determined in accordance with methods and tools well known to those of skill in the art. One tool for measuring the osmolality is a vapor pressure osmometer (e.g., VAPRO vapor pressure osmometer from ELITechGroup, Inc., Puteaux, France). In embodiments, the oral rehydration slush composition of the present disclosure has an osmolality of 100 mOsm/kg H₂O to 325 mOsm/kg H₂O. In embodiments, the oral rehydration slush composition of the present disclosure has an osmolality of 150 mOsm/kg H₂O to 320 mOsm/kg H₂O. In embodiments, the oral rehydration slush composition of the present disclosure has an osmolality of 200 mOsm/kg H₂O to 315 mOsm/kg H₂O. In embodiments, the oral rehydration slush composition of the present disclosure has an osmolality of 250 mOsm/kg H₂O to 310 mOsm/kg H₂O. In embodiments, the oral rehydration slush composition of the present disclosure has an osmolality of 285 mOsm/kg H₂O to 305 mOsm/kg H₂O. In embodiments, the oral rehydration slush composition of the present disclosure has an osmolality of 290 mOsm/kg H₂O to 305 mOsm/kg H₂O.

The oral rehydration slush composition of the present disclosure may also include a gum system. As mentioned above, the incorporation of a gum system provides an oral rehydration slush composition having a slush texture and consistency similar to that of a conventional slush composition, while maintaining a low osmolality (i.e., less than or equal to 350 mOsm/kg H₂O) so that the oral rehydration slush composition is suitable for oral rehydration therapy.

In embodiments, the oral rehydration slush composition of the present disclosure comprises a gum system comprising at least one of a water binding gum and an emulsifying gum. In embodiments, the oral rehydration slush composition of the present disclosure comprises a gum system comprising a water binding gum. In embodiments, the oral rehydration slush composition of the present disclosure comprises a gum system comprising a water binding gum and an emulsifying gum.

Exemplary water binding gums suitable for use in the oral rehydration slush composition of the present disclosure include, but are not limited to, guar gum, carboxymethyl cellulose, hydroxypropyl methylcellulose, methyl cellulose, locust bean gum, tara gum, xanthan gum, and combinations thereof. One example of an emulsifying gum suitable for use in the oral rehydration slush composition of the present disclosure is gum acacia. In embodiments, the oral rehydration slush composition includes a gum system comprising guar gum. In embodiments, the oral rehydration slush composition includes a gum system comprising guar gum and gum acacia.

In embodiments, the oral rehydration slush composition comprises a gum system in an amount of 0.25 wt % to 0.75 wt %, based on the total weight of the oral rehydration slush composition. In embodiments, the oral rehydration slush composition comprises a gum system in an amount of 0.3 wt % to 0.6 wt %, based on the total weight of the oral rehydration slush composition. In embodiments, the oral rehydration slush composition comprises a gum system in an amount of 0.35 wt % to 0.5 wt %, based on the total weight of the oral rehydration slush composition. In embodiments, the oral rehydration slush composition comprises a gum system in an amount of 0.35 wt % to 0.45 wt %, based on the total weight of the oral rehydration slush composition. In certain of the foregoing embodiments, the gum system comprises guar gum. In certain of the foregoing embodiments, the gum system comprises guar gum and gum acacia. In certain of the foregoing embodiments, the gum system comprises at least 50 wt % guar gum and less than 50 wt % gum acacia, based on the total weight of the gum system. One example of a gum system suitable for use in the oral rehydration slush composition of the present disclosure is TIC Pretested® Stabilizer ICE-200 Powder, which is commercially available from TIC Gums, Inc. (Belcamp, Md.).

The oral rehydration slush composition of the present disclosure has a slush texture and consistency similar to that of a conventional slush composition while maintaining a low osmolality (i.e., less than or equal to 350 mOsm/kg H₂O). The slush texture and consistency may be characterized by ice crystal particle size, particularly the D90 particle size. As one of skill in the art knows, the D90 particle size refers to the particle size at which 90% of the particle size distribution has a smaller particle size and 10% of the particle size distribution has a larger particle size. In embodiments, the oral rehydration slush composition has a D90 particle size of 15 microns to 35 microns in a ready-to-eat frozen state. In embodiments, the oral rehydration slush composition has a D90 particle size of 20 microns to 30 microns in a ready-to-eat frozen state.

The phrase “ready-to-eat frozen state” refers to the state achieved when the oral rehydration slush composition is prepared using a commercially available frozen beverage machine, such as an Ultra-2 HP Liquid Autofill Frozen Beverage System (PN: 34000.0176) from Bunn-O-Matic Corporation (Springfield, Ill.). An oral rehydration slush composition in a ready-to-eat frozen state may be prepared in accordance with the following procedure using the Ultra-2 HP Liquid Autofill Frozen Beverage System. First, the dry ingredients of the oral rehydration slush composition are added to the appropriate amount of room temperature (e.g., 15° C. to 24° C.) water and are mixed until the ingredients are dissolved to form an aqueous mixture. Alternatively, a packaged aqueous mixture including the ingredients of the slush composition can be used. Next, the room temperature aqueous mixture is transferred to a cooler (at about 5° C.) for at least six hours. After cooling, the aqueous mixture is transferred to a hopper of the frozen beverage machine. After transferring the cooled aqueous mixture to the hopper, the frozen beverage machine is powered on. Next, the auger motor for the hopper containing the cooled aqueous mixture is powered on. The “THICKNESS” setting for the frozen beverage machine is maintained at the factory default setting of 10. The hopper containing the cooled aqueous mixture is put in “ICE” mode to begin the freezing process. The hopper is left in ICE mode with the auger motor powered on for at least 1 hour to render the oral rehydration slush composition in a ready-to-eat frozen state.

The D90 particle size of the oral rehydration slush composition may be determined in accordance with methods well known to those of skill in the art. One example of such a method is focused beam reflectance measurement.

Unlike conventional slush compositions, the oral rehydration slush composition of the present disclosure does not include common organic compounds used as freezing point depression agents that are classified as alcohols (e.g., glycerol, monopropylene glycol, ethanol) to achieve the desired slush texture and consistency. In embodiments, the oral rehydration slush composition is free of glycerol. In embodiments, the oral rehydration slush composition is free of glycerol and/or monopropylene glycol.

The oral rehydration slush composition of the present disclosure may include one or more additional ingredients. Examples of additional ingredients in the oral rehydration slush composition of the present disclosure include, but are not limited to, flavorants, colorants, vitamins, additional minerals, and combinations thereof. In general, the amount of any additional ingredients in the oral rehydration slush composition of the present disclosure is less than 0.25 wt %, based on the total weight of the oral rehydration slush composition, including less than 0.1 wt %, based on the total weight of the oral rehydration slush composition.

In embodiments, a flavorant may be present to add or modify a flavor of the oral rehydration slush composition. Examples of suitable flavorants include, but are not limited to, blue raspberry flavorant, cherry flavorant, strawberry flavorant, lemon flavorant, orange flavorant, grape flavorant, fruit punch flavorant, bubble gum flavorant, lime flavorant, and combinations thereof. The flavorants may be a natural flavorant, an artificial flavorant, or a combination of a natural flavorant and an artificial flavorant.

In embodiments, artificial sweeteners may also be added to complement the flavor of the oral rehydration slush composition. Exemplary artificial sweeteners include, but are not limited to, sucralose, acesulfame-K (ace-K), saccharin, aspartame, and the like.

In embodiments, a colorant may be present to add or modify a color of the oral rehydration slush composition. Exemplary colorants suitable for use in the oral rehydration slush composition of the present disclosure include, but are not limited to, FD&C Red No. 40, FD&C Red No. 3, FD&C Red No. 20, FD&C Yellow No. 6, FD&C Blue No. 1, FD&C Blue No. 2, FD&C Green No. 5, FD&C Orange No. 5, FD&C Red No. 8, red beet powder, beta carotene, caramel, grape skin extract, turmeric, paprika, and the like, and combinations thereof.

In an embodiment, the oral rehydration slush composition of the present disclosure comprises: water; dextrose; 15 mEq/L to 120 mEq/L of sodium; 2 mEq/L to 60 mEq/L of potassium; 25 mEq/L to 110 mEq/L of chloride; and 5 mmol/L to 40 mmol/L of citrate. The oral rehydration slush composition also has an osmolality of 70 mOsm/kg H₂O to 350 mOsm/kg H₂O. The sodium, potassium, chloride, and citrate may be provided by any of the previously described sources of sodium, potassium, chloride, and citrate. This embodiment of the oral rehydration slush composition may also include a gum system (e.g., gum system comprising guar gum or gum system comprising guar gum and gum acacia) as previously described herein. In addition, the previously described amounts of water and other ingredients, total solids content, and D90 particle size apply equally to this embodiment.

In another aspect of the present disclosure, a method of treating dehydration in an individual in need thereof is provided. The individual may be afflicted with dehydration or otherwise in need of hydration for any number of reasons. The method of treating dehydration includes administering to the individual in need thereof an oral rehydration slush composition. The oral rehydration slush composition may be any one of the compositions described herein. In embodiments, the individual in need thereof requires hydration due to heat exposure, exercise, illness, vomiting, diarrhea, and surgery recovery. In embodiments, the individual in need thereof may be a child (ages 1 to 18), an adult (age 18+), an elderly adult (age 65+), an individual suffering from cancer, an individual suffering from dysphagia, or an individual recovering from surgery.

The oral rehydration slush composition of the present disclosure can supply an individual afflicted with dehydration or otherwise in need of hydration the vital fluids and necessary electrolytes that the individual would otherwise be unable to absorb. This is accomplished through a balance between the amount of carbohydrates and the amount of electrolytes in the oral rehydration slush composition. In embodiments, the oral rehydration slush composition includes a source of carbohydrate comprising dextrose and a source of electrolytes comprising a source of sodium, and a molar ratio of dextrose to sodium is 1:1 to 3:1. Such a ratio of dextrose to sodium improves an individual's ability to absorb sodium.

The oral rehydration slush compositions of the present disclosure may be made by adding a source of carbohydrate, a source of electrolytes, and a source of citrates to an appropriate amount of water. In embodiments, a gum system as previously described may be added along with the other ingredients to the water. The water and ingredients are mixed together until the ingredients dissolve forming an aqueous mixture. The aqueous mixture may be aseptically, retort, or hot-fill packaged in single-serve pouches or other appropriate packaging. The packaged aqueous mixture may be quiescently frozen by subjecting the packaged aqueous mixture to a temperature of less than or equal to 0° C. for a predetermined period of time (e.g., at least 1 hour, at least 6 hours, at least 24 hours). When ready to be consumed, the frozen, packaged aqueous mixture may be allowed to partially thaw at room temperature (e.g., 15° C. to 24° C.) and manipulated by hand (e.g., by squeezing or massaging the partially thawed contents of the package) to render the oral rehydration slush composition. Alternatively, the contents of the packaged aqueous mixture may be added to a conventional frozen beverage machine to render the oral rehydration slush composition, as previously described.

In embodiments, the oral rehydration slush composition of the present disclosure is provided in powder form. In other words, the dry ingredients (i.e., all ingredients but water) of the oral rehydration slush composition, such as a source of carbohydrate, a source of electrolytes, a source of citrate, and a gum system, are provided in powder form, which may be packaged in single-serve packaging (e.g., a stick pouch) or a bulk container holding multiple servings. The oral rehydration slush composition powder may then be added to and mixed with the appropriate amount of water and frozen to render the oral rehydration slush composition in a ready-to-eat form. In embodiments, the oral rehydration slush composition powder comprises 35 wt % to 65 wt % dextrose, 10 wt % to 20 wt % citric acid, 10 wt % to 18 wt % gum system, 5 wt % to 13 wt % sodium chloride, 2 wt % to 4 wt % sodium citrate, and 1 wt % to 3 wt % flavorants and sweeteners, where each weight percentage is based on the total weight of the oral rehydration slush composition powder. In embodiments, the oral rehydration slush composition powder has a total dry solids content of 97% to 100%.

### Example

The following example further describes and demonstrates specific embodiments within the scope of the present disclosure. The example is for purposes of illustration only and is not intended to limit the scope of the present disclosure.

Table 1 shows the ingredients used to form four slush compositions (i.e., Slushie 1, Slushie 2, Slushie 3, and Slushie 4). Slushie 2 and Slushie 3 illustrate exemplary embodiments of ingredients used to form an oral rehydration slush composition in accordance with the present disclosure. The ingredients used to form Slushie 1 correspond to a commercially available ready-to-drink oral rehydration product. The ingredients used to form Slushie 4 correspond to a conventional slush composition.

TABLE 1

| | Slushie 1 | Slushie 2 | Slushie 3 | Slushie 4 |
|---|---|---|---|---|
| INGREDIENTS | Weight % | Weight % | Weight % | Weight % |
| Water | 96.5 | 96.0 | 96.0 | 85.4 |
| Glycerol | — | — | — | 11.2 |
| Dextrose | 2.5 | 2.5 | 2.5 | 2.5 |
| Citric Acid | 0.3 | 0.5 | 0.5 | 0.3 |
| Guar Gum | — | 0.4 | — | — |
| Guar Gum + | — | — | 0.4 | — |
| Gum Acacia Blend | | | | |
| Potassium Citrate | 0.2 | 0.2 | 0.2 | 0.2 |
| Sodium Chloride | 0.2 | 0.2 | 0.2 | 0.2 |
| Sodium Citrate | 0.1 | 0.1 | 0.01 | 0.1 |
| Flavor + Sweeteners | 0.05 | 0.08 | 0.08 | 0.05 |

The method used to make each slush composition was identical. First, all ingredients were added to the appropriate amount of room temperature (e.g., 15° C. to 24° C.) water and mixed until the ingredients were dissolved to form an aqueous mixture. Next, the room temperature aqueous mixture was transferred to a cooler (at about 5° C.) for at least six hours. After cooling, the aqueous mixture was transferred to a hopper of a commercial frozen beverage machine. In this example, an Ultra-2 HP Liquid Autofill Frozen Beverage System (PN: 34000.0176) from Bunn-O-Matic Corporation (Springfield, Ill.) was utilized. After transferring the cooled aqueous mixture to the hopper, the frozen beverage machine was powered on. Next, the auger motor for the hopper containing the cooled aqueous mixture was powered on. The “THICKNESS” setting for the frozen beverage machine was left at the factory default setting of 10. The hopper containing the cooled aqueous mixture was then put in “ICE” mode to begin the freezing process. For each slush composition, the hopper was left in ICE mode with the auger motor powered on for at least 6 hours.

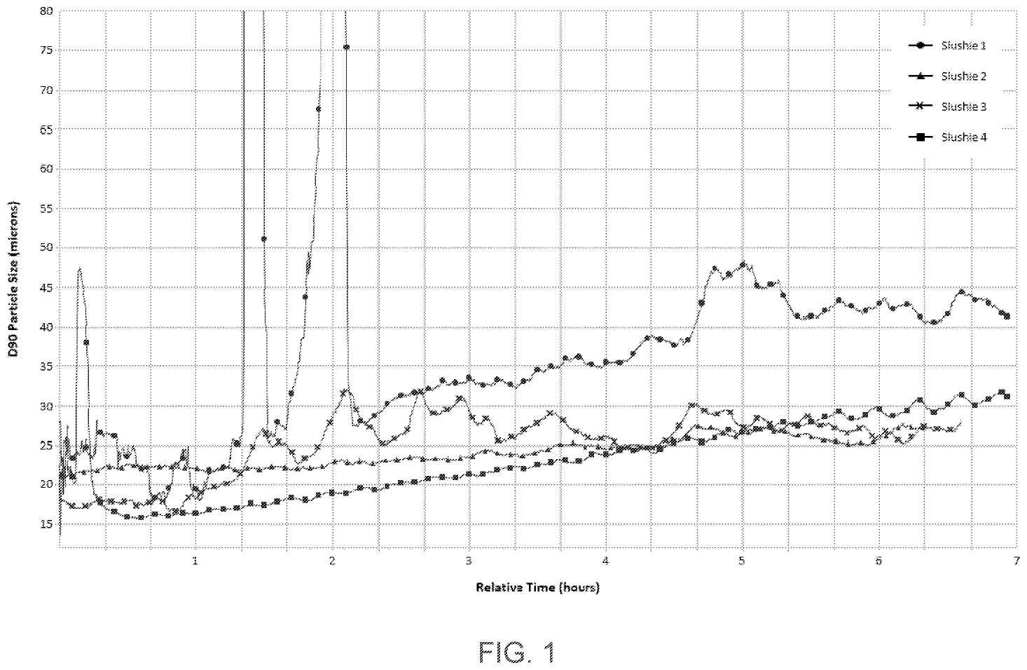

Ice crystal formation and growth throughout the freezing process were monitored via focused beam reflectance measurement (FBRM) for each slush composition immediately after ICE mode was turned on. The FBRM was used to collect D90 particle size data of the ice crystals present in each slush composition. The D90 particle size for each slush composition during the freezing process is illustrated graphically in FIG. 1. In addition, the osmolality of each slush composition (in liquid form) was measured using a vapor pressure osmometer. The osmolality for Slushie 1 was 270 mOsm/kg H₂O. The osmolality for Slushie 2 and Slushie 3 were 300 mOsm/kg H₂O and 290 mOsm/kg H₂O, respectively. The osmolality for Slushie 4 was 1,740 mOsm/kg H₂O.

As seen in the FIGURE, throughout the freezing process, the D90 particle size trends of Slushie 2 and Slushie 3 are more consistent with the D90 particle size trend of Slushie 4 than Slushie 1. It can be seen from the osmolality data and D90 particle size data that the addition of a gum system enables the production of a low osmolality oral rehydration slush composition that has a slush texture and consistency comparable to that of a conventional slush composition, which primarily relies on freezing point depression via high product osmolality.

Unless otherwise indicated herein, all sub-embodiments and optional embodiments are respective sub-embodiments and optional embodiments to all embodiments described herein. While the present disclosure has been illustrated by the description of embodiments thereof, and while the embodiments have been described in considerable detail, it is not the intention of the applicant to restrict or in any way limit the scope of the appended claims to such detail. Additional advantages and modifications will readily appear to those skilled in the art. Therefore, the present disclosure, in its broader aspects, is not limited to the specific details, the representative compositions or formulations, and illustrative examples shown and described. Accordingly, departures may be made from such details without departing from the spirit or scope of Applicant's general disclosure herein.

What is claimed is:

1. An oral rehydration slush composition comprising:
water;
dextrose;
15 mEq/L to 120 mEq/L of sodium;
2 mEq/L to 60 mEq/L of potassium;
25 mEq/L to 110 mEq/L of chloride; and
5 mmol/L to 40 mmol/L of citrate,
wherein the oral rehydration slush composition has an osmolality of 70 mOsm/kg H₂O to 350 mOsm/kg H₂O.

2. The oral rehydration slush composition of claim 1, further comprising a gum system comprising at least one of a water binding gum and an emulsifying gum.

3. The oral rehydration slush composition of claim 2, wherein the water binding gum comprises at least one of carboxymethyl cellulose, hydroxypropyl methylcellulose, methyl cellulose, guar gum, locust bean gum, tara gum, xanthan gum, and combinations thereof, and the emulsifying gum comprises gum acacia.

4. The oral rehydration slush composition of claim 2, wherein the gum system comprises 0.25 wt % to 0.75 wt % of the oral rehydration slush composition.

5. The oral rehydration slush composition of claim 1, wherein a molar ratio of dextrose to sodium is 1:1 to 3:1.

6. The oral rehydration slush composition of claim 1, wherein the sodium is provided by at least one of sodium chloride, sodium phosphate, sodium citrate, sodium carbonate, sodium bicarbonate, sodium hydroxide, and combinations thereof,
wherein the potassium is provided by at least one of potassium chloride, potassium phosphate, potassium citrate, potassium carbonate, potassium bicarbonate, potassium hydroxide, and combinations thereof, and
wherein the chloride is provided by at least one of sodium chloride, potassium chloride, calcium chloride, magnesium chloride, and combinations thereof.

7. The oral rehydration slush composition of claim 1, wherein the oral rehydration slush composition has a total solids content of 2% to 10%

8. The oral rehydration slush composition of claim 1, wherein the oral rehydration slush composition is free of glycerol.

9. An oral rehydration slush composition comprising:
water;
a source of carbohydrate;
a source of electrolytes; and
a source of citrate,
wherein the oral rehydration slush composition has an osmolality of 70 mOsm/kg H₂O to 350 mOsm/kg H₂O.

10. The oral rehydration slush composition of claim 9, further comprising a gum system comprising at least one of a water binding gum and an emulsifying gum.

11. The oral rehydration slush composition of claim 10, wherein the water binding gum comprises at least one of carboxymethyl cellulose, hydroxypropyl methylcellulose, methyl cellulose, guar gum, locust bean gum, tara gum, xanthan gum, and combinations thereof, and the emulsifying gum comprises gum acacia.

12. The oral rehydration slush composition of claim 10, wherein the gum system comprises 0.25 wt % to 0.75 wt % of the total weight of the oral rehydration slush composition.

13. The oral rehydration slush composition of claim 9, wherein the source of electrolytes comprises one or more of a source of sodium, a source of potassium, and a source of chloride.

14. The oral rehydration slush composition of claim 13, wherein the source of sodium is present in an amount sufficient to provide 15 mEq/L to 120 mEq/L of sodium, wherein the source of potassium is present in an amount sufficient to provide 2 mEq/L to 60 mEq/L of potassium, and wherein the source of chloride is present in an amount sufficient to provide 25 mEq/L to 110 mEq/L of chloride.

15. The oral rehydration slush composition of claim 13, wherein the source of carbohydrate comprises dextrose, and a molar ratio of dextrose to sodium is 1:1 to 3:1.

16. The oral rehydration slush composition of claim 9, wherein the source of citrate is present in an amount sufficient to provide 5 mmol/L to 40 mmol/L of citrate.

17. The oral rehydration slush composition of claim 13, wherein the source of sodium is at least one of sodium chloride, sodium phosphate, sodium citrate, sodium carbonate, sodium bicarbonate, sodium hydroxide, and combinations thereof,
wherein the source of potassium is at least one of potassium chloride, potassium phosphate, potassium citrate, potassium carbonate, potassium bicarbonate, potassium hydroxide, and combinations thereof, and
wherein the source of chloride is at least one of sodium chloride, potassium chloride, calcium chloride, magnesium chloride, and combinations thereof.

18. The oral rehydration slush composition of claim 9, wherein the oral rehydration slush composition has a total solids content of 2% to 10%.

19. (canceled)

20. The oral rehydration slush composition of claim 1, wherein the oral rehydration slush composition has a D90 particle size of 15 microns to 35 microns in a ready-to-eat frozen state.

21. A method of treating dehydration in an individual in need thereof, the method comprising administering to the individual in need thereof the oral rehydration slush composition of claim 1.
